(19) United States
(12) Patent Application Publication
(10) Pub. No.: US 2008/0268018 A1
(43) Pub. Date: Oct. 30, 2008

(54) Method for forming crystallized therapeutic agents on a medical device

(76) Inventor: Stephen D. Pacetti, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/799,263

(22) Filed: Apr. 30, 2007

(51) Int. Cl. A61K 47/00; A61P 9/00; A61P 9/10

(52) U.S. Cl. 424/426; 424/423

(57) ABSTRACT

A method of crystallizing a therapeutic agent in a coating on an implantable medical device, and uses thereof, are disclosed.

### FIELD OF THE INVENTION

The present invention relates to a method for forming crystallized therapeutic agents in a coating on a medical device, and methods of using the device for treating a vascular disease.

### BACKGROUND OF THE INVENTION

The traditional method of administering therapeutic agents to treat diseases of the internal organs and vasculature has been by systemic delivery. Systemic delivery involves administering a therapeutic agent at a discrete location followed by the agent migrating throughout the patient's body including, of course, to the afflicted organ or area of the vasculature. But to achieve a therapeutic amount of the agent at the afflicted site, an initial dose substantially greater than the therapeutic amount must be administered to account for the dilution the agent undergoes as it travels through the body.

At the other end of the spectrum is local delivery, which comprises administering the therapeutic agent directly to the afflicted site. With localized delivery, the initial dose can be at or very close to the therapeutic amount. With time, some of the locally delivered therapeutic agent may diffuse over a wider region, but that is not the intent of localized delivery, and the diffused portion's concentration will ordinarily be sub-therapeutic, i.e., too low to have a therapeutic effect. Nevertheless, localized delivery of therapeutic agents is currently considered a state-of-the-art approach to the treatment of many diseases such as, without limitation, cancer and atherosclerosis.

Localized delivery of therapeutic agents includes the use of coated implantable medical devices, e.g., a drug delivery stent. A drug delivery stent can be positioned at an afflicted site within the vasculature thereby allowing the direct administration of a drug to a vascular site in need thereof.

Drug delivery stents can also be designed to release more than one drug. For example, stents can be coated with both an anti-proliferative drug, e.g., everolimus, and an anti-inflammatory drug, e.g., dexamethasone.

In certain situations, a selected drug may crystallize in the stent coating. While this phenomenon is not necessarily bad, if the crystallization is inconsistent, it can lead to release rate variability. Moreover, if the degree of crystallization changes with time, a drift in release rate over time could also occur. Indeed, if the crystals form on the surface of the coating, as opposed to the interior, an embolic hazard may present.

This present invention solves these problems, among others, by promoting uniform, thorough drug crystallization on implantable medical devices by the intentional addition of micro- or nano-sized particles to a medical device coating formulation. The micro- and nano-sized particles are insoluble in the coating formulation and serve as crystallization nuclei to promote uniform and complete crystallization.

### SUMMARY OF THE INVENTION

The present invention relates to a method of crystallizing a therapeutic agent in a coating on an implantable medical device. The method involves providing an implantable medical device, providing a coating formulation that includes a solvent, one or more polymers dissolved in the solvent, one or more crystallizable therapeutic agents dissolved in the solvent and a plurality of non-soluble nucleation particles suspended in the solvent, coating the implantable medical device with the coating formulation and drying the coating. The implantable medical device can be a stent.

In one aspect, the solvent is an organic solvent.

In various aspects, the one or more crystallizable therapeutic agents are selected from the group consisting of an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist and an antioxidant. In various embodiments, the anti-inflammatory agent can be dexamethasone, clobestasol, momentasone, dexamethasone acetate, cortisone, prednisone, prednisolone or betamethasone.

In various aspects, the plurality of non-soluble nucleation particles can include a pharmaceutical excipient, a biodegradable polymer or a GRAS material.

In various aspects, the plurality of non-soluble nucleation particles is non-toxic and dissolves upon release from the coating or dissolves within the coating.

In various aspects, the plurality of non-soluble nucleation particles can have a maximum linear dimension of 2 microns, a maximum linear dimension of 300 nanometers, a maximum linear dimension of 100 nanometers or a maximum linear dimension of 10 nanometers. The population of non-soluble nucleation particles can be monodisperse or can include a distribution of sizes spanning the above dimensions.

In one aspect, the plurality of non-soluble nucleation particles have a maximum linear dimension no greater than 1/10 the final thickness of the coating.

In one aspect, the weight of nucleation particles added to the coating formulation is less than 25 percent of the weight of crystallizable therapeutic agent added to the coating formulation.

In one aspect, the crystallized therapeutic agent enhances the stability of the coated implantable medical device during aging.

In one aspect, the crystallized therapeutic agent is uniformly released from the coated implantable medical device after implantation.

Another aspect of the present invention relates to a method of treating or preventing a vascular disease. The method involves providing an implantable medical device of the invention and implanting the implantable medical device in a vessel of a patient in need thereof.

The vascular disease to be treated can be atherosclerosis, restenosis, vulnerable plaque or peripheral arterial disease.

Another aspect of the present invention relates to a method for controlling the release rate of a therapeutic agent from an implantable medical device. The method involves providing an implantable medical device, coating the implantable medical device with a formulation comprising a solvent, one or more polymers dissolved in the solvent, one or more crystallizable therapeutic agents dissolved in the solvent and a plurality of non-soluble nucleation particles suspended in the solvent and drying the coating.

In one aspect, the implantable medical device can be a stent.

In one aspect, the solvent is an organic solvent.

In various aspects, the one or more crystallizable therapeutic agents are selected from the group consisting of an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist and an antioxidant.

In various aspects, the plurality of non-soluble nucleation particles includes a pharmaceutical excipient, a biodegradable polymer or a GRAS material.

In one aspect, the plurality of non-soluble nucleation particles have a maximum linear dimension of 2 microns.

In one aspect, the crystallized therapeutic agent is uniformly released from the coated implantable medical device after implantation.

### BRIEF DESCRIPTION OF THE DRAWINGS

FIG. 1 is an optical micrograph under crossed polarizers showing a 100 μg/cm² everolimus, 50 μg/cm² dexamethasone coating at a drug to polymer ratio of 1 to 7 (w/w), where 100 μg/cm² indicates a dose of 100 μg of drug per cm² of stent surface area.

### DETAILED DESCRIPTION OF THE INVENTION

The present invention relates to a method of crystallizing a therapeutic agent in a coating on an implantable medical device. The method involves providing an implantable medical device and providing a coating formulation that includes a solvent, one or more polymers dissolved in the solvent, one or more crystallizable therapeutic agents dissolved in the solvent and a plurality of non-soluble nucleation particles suspended in the solvent. The implantable medical device is coated with the coating formulation, then the coating is dried. The method facilitates uniform and rapid crystallization of the therapeutic agent by nucleation, resulting in a stable and uniform therapeutic agent coating for use on implantable medical devices.

Suitable implantable medical devices include, but are not limited to, stents, stent-grafts, vascular grafts, artificial heart valves, foramen ovale closure devices, cerebrospinal fluid shunts, pacemaker electrodes, guidewires, ventricular assist devices, cardiopulmonary bypass circuits, blood oxygenators, coronary shunts (AXIUS™, Guidant Corp.), vena cava filters, and endocardial leads (FINELINE® and ENDOTAK®, Guidant Corp.). In some embodiments, the stents include, but are not limited to, tubular stents, self-expanding stents, coil stents, ring stents, multi-design stents and the like. In other embodiments, the stents are metallic, low-ferromagnetic, non-ferromagnetic, biostable polymeric, biodegradable polymeric or biodegradable metallic. In some embodiments, the stents include, but are not limited to, vascular stents, renal stents, biliary stents, pulmonary stents, urethral stents and gastrointestinal stents.

Biostable refers to polymers that are not degraded in vivo. The terms bioabsorbable, biodegradable and bioerodable, as well as absorbed, degraded and eroded are use interchangeably (unless the context show otherwise) and refer to polymers and metals that are capable of being degraded or absorbed after being delivered to a disease locale in a patient, e.g., when exposed to bodily fluids such as blood, and that can be gradually resorbed, absorbed and/or eliminated by the body.

A suitable solvent for use in the coating formulation is chosen based on several criteria including, for example, its polarity, ability to hydrogen bond, molecular size, volatility, biocompatibility, reactivity and purity. The choice of solvent, however, is primarily determined by the choice of therapeutic agent and nucleation particle because in order for the nucleation particle to act as a nucleus for the crystallization of the therapeutic agent, it must be insoluble in the chosen solvent. In addition, the solvent must dissolve the coating polymer of interest. Methods of choosing a suitable solvent are known to those skilled in the art.

Potentially suitable solvents for use in the present invention include, but are not limited to, dimethyl acetamide (DMAC), dimethyl formamide (DMF), tetrahydrofuran (THF), TCE (1,1,2,2-tetrachloroethane), acetone, Dowanol™ (2-(2-ethoxyethoxy)ethanol), DCM (dichloromethane), MEK (methyl ethyl ketone), chloroform, ethanol, butanol, isopropyl acetate, pentane. Other solvents that can be used include, but are not limited to, cyclohexanone, xylene, toluene, propylene glycol monomethyl ether, methyl butyl ketone, ethyl acetate, n-butyl acetate, and dioxane. Solvent mixtures can be used as well. Examples of the mixtures include, but are not limited to, DMAC and methanol (50:50 w/w); water, i-propanol, and DMAC (10:3:87 w/w); i-propanol and DMAC (80:20, 50:50, or 20:80 w/w); acetone and cyclohexanone (80:20, 50:50, or 20:80 w/w); acetone and xylene (50:50 w/w); acetone, xylene and FLUX REMOVER AMS® (93.7% 3,3-dichloro-1,1,1,2,2-pentafluoropropane and 1,3-dichloro-1,1,2,2,3-pentafluoropropane, and the balance is methanol with trace amounts of nitromethane; Tech Spray, Inc.) (10:40:50 w/w); and TCE and chloroform (80:20 w/w). Preferably the solvent is an organic solvent.

Suitable polymers useful in the present invention can be biodegradable or non-biodegradable and can be hydrophobic or hydrophilic. Suitable polymers include, but are not limited to, poly(ester amide), poly(ethylene-co-vinyl alcohol) (commonly known by the generic name EVOH or by the trade name EVAL), poly(L-lactic acid) (PLLA), poly(L-lactide), poly(D,L-lactide), poly(L-lactide-co-D,L-lactide), polycaprolactone (PCL), poly(lactide-co-glycolide), poly(hydroxybutyrate), poly(hydroxybutyrate-co-valerate), polydioxanone, polyorthoester, polyanhydride, poly(glycolic acid) (PGA), poly(D,L-lactic acid) (PDLLA), poly(D,L-lactide-co-glycolide) (PDLLAGA), poly(glycolic acid-co-trimethylene carbonate), poly(D-lactic acid) (PDLA), poly(D,L-lactic acid-co-glycolic acid) (PDLLGA), polyhydroxyalkanoates (PHA), poly(3-hydroxybutyrate) (PHB), poly(3-hydroxybutyrate-co-3-hydroxyvalerate), poly((3-hydroxyvalerate), poly(3-hydroxyhexanoate), poly(4-hydroxybutyrate), poly(4-hydroxyvalerate), poly(4-hydroxyhexanoate), PEG-PLA, PCL-PLA where the monomer lactic acid can be either a D- or L-stereo isomer, a racemic mixture or a blend of the D- and L-isomer, poly(urethanes), polyphosphoester, polyphosphoester urethane, poly(amino acids), polycyanoacrylates, poly(trimethylene carbonate), poly(iminocarbonate), poly(butylene terephthalate-co-poly(ethylene glycol) (PEG)-terephthalate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides such as vinylidene fluoride based homo or copolymers under the trade name Solef™ or Kynar™, for example, polyvinylidene fluoride (PVDF) or poly(vinylidene-co-hexafluoropropylene) (PVDF-co-HFP) and polyvinylidene chloride, polyacrylonitrile, polyvinyl ketones, polyvinyl aromatics such as polystyrene, polyvinyl esters, such as polyvinyl acetate, copolymers of vinyl monomers with each other and olefins such as ethylene-methyl methacrylate copolymers, acrylonitrile-styrene copolymers, ABS resins, and ethylene-vinyl acetate copolymers, polyamides such as Nylon 66 and polycaprolactam, alkyd resins, polycarbonates, polyoxymethylenes, polyimides, polyethers, poly(glyceryl sebacate), poly(propylene fumarate), epoxy resins, polyurethanes, rayon, rayon-triacetate, cellulose acetate, cellulose butyrate, cellulose acetate butyrate, cellophane, cellulose nitrate, cellulose propionate, cellulose ethers and carboxymethyl cellulose, or a combination thereof.

Therapeutic agents are primarily chosen for their therapeutic properties. Often, however, a therapeutic agent will crystallize in the selected solvent/polymer mix. If the crystallization process is not reproducible or the rate of crystallization varies over time, release rate variability can occur. Moreover, if crystals form on an implantable medical device coating surface an embolic hazard may present. The methods of the present invention solve these problems by promoting uniform, thorough drug crystallization on implantable medical devices which, among other things, promotes greater agent stability and a prolonged agent release profile.

Suitable crystallizable therapeutic agents include, but are not limited to, an antiproliferative agent, an anti-inflammatory agent, a steroid, a glucocorticoid, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist and an antioxidant. In various embodiments, the crystallizable therapeutic agent is dexamethasone, clobestasol, momentasone, dexamethasone acetate, cortisone, prednisone, prednisolone, betamethasone, paclitaxel or cisplatin.

In various embodiments, the plurality of non-soluble nucleation particles can include a pharmaceutical excipient, a biodegradable polymer or a GRAS material.

As used herein, “non-soluble” refers to the inability of nucleation particles to dissolve in a given solvent.

As used herein, “excipient” refers to a chemically inert substance.

Suitable pharmaceutical excipients include, without limitation, magnesium stearate, lactose, microcrystalline cellulose, starch (corn), silicon dioxide, titanium dioxide, stearic acid, sodium starch glycolate, gelatin, talc, sucrose, calcium stearate, pregelatinized starch, hydroxy propyl methylcellulose, croscarmellose, hydroxy propyl cellulose, ethylcellulose, calcium phosphate (dibasic).

In another embodiment, the non-soluble nucleation particles include a biodegradable polymer. Because many biodegradable polymers are soluble in organic solvents, their use as non-soluble nucleation particles may be limited to water- or alcohol-based coating formulations. Suitable biodegradable polymers are described above.

In another embodiment, the non-soluble nucleation particles can be made of a material ‘generally recognized as safe’ by the Food and Drug Administration, i.e., a GRAS list material. GRAS list materials include materials used as food additives. Particles of these materials are also encompassed by the present invention. Suitable GRAS list materials include, without limitation, aluminum calcium silicate, calcium silicate, magnesium silicate, sodium aluminosilicate, sodium calcium aluminosilicate, tricalcium silicate, ascorbic acid, ascorbyl palmitate, benzoic acid, butylated hydroxyanisole, butylated hydroxytoluene, calcium ascorbate, calcium propionate, calcium sorbate, caprylic acid, dilauryl thiodipropionate, erythorbic acid, gum guaiac, methylparaben, potassium bisulfite, potassium metabisulfite, potassium sorbate, propionic acid, propyl gallate, propylparaben, sodium ascorbate, sodium benzoate, sodium bisulfite, sodium metahisulfite, sodium propionate, sodium sorbate, sodium sulfite, sorbic acid, stannous chloride, thiodipropionic acid, tocopherols, cholic acid, desoxycholic acid, diacetyl tartaric acid esters of mono- and di-glycerides, glycocholic acid, mono- and diglycerides, monosodium phosphate, taurocholic acid, alanine, arginine, ascorbic acid, aspartic acid, biotin, calcium carbonate, calcium citrate, calcium glycerophosphate, calcium oxide, calcium pantothenate, calcium phosphate, calcium pyrophosphate, calcium sulfate, carotene, choline bitartrate, choline chloride, copper gluconate, cuprous iodide, cysteine, cystine, ferric phosphate, ferric pyrophosphate, ferric sodium pyrophosphate, ferrous gluconate, ferrous lactate, ferrous sulfate, glycine, histidine, inositol and iron.

Other suitable GRAS list materials include isoleucine, leucine, linoleic acid, lysine, magnesium oxide, magnesium phosphate, magnesium sulfate, manganese chloride, manganese citrate, manganese gluconate, manganese glycerophosphate, manganese hypophosphate, manganese sulfate, manganous oxide, mannitol, methionine, methionine hydroxy analogue, niacin, niacinamide, D-pantothenyl alcohol, phenylalanine, potassium chloride, potassium glycerophosphate, potassium iodide, praline, pyridoxine hydrochloride, riboflavin, riboflavin-5-phosphate, serine, sodium pantothenate, sodium phosphate, sorbitol, thiamine hydrochloride, thiamine mononitrate, threonine, tocopherols, tocopherol acetate, tyrosine, valine, vitamin A, vitamin A acetate, vitamin A palmitate, vitamin B12, vitamin D2, vitamin D3, zinc sulfate, zinc gluconate, zinc chloride, zinc oxide, zinc stearate, calcium acetate, calcium chloride, calcium citrate, calcium diacetate, calcium gluconate, calcium hexametaphosphate, calcium phosphate-monobasic, calcium phytate, citric acid, dipotassium phosphate, disodium phosphate, isopropyl citrate, monoisopropyl citrate, potassium citrate, sodium acid phosphate, sodium citrate, sodium diacetate, sodium gluconate, sodium hexametaphosphate, sodium metaphosphate, sodium phosphate, sodium potassium tartrate, sodium pyrophosphate, sodium pyrophosphate-tetra, sodium tartrate, sodium thiosulfate, sodium tripolyphosphate, tartaric acid, acacia (gum arabic), agar, ammonium alginate, calcium alginate, carob bean gum, chondrus extract, ghatti gum, guar gum, potassium alginate, sodium alginate, sterculia (or Karava) gum, tragacanth, acetic acid, adipic acid, aluminum ammonium sulfate, aluminum potassium sulfate, aluminum sodium sulfate, aluminum sulfate, ammonium bicarbonate, ammonium carbonate, ammonium hydroxide, ammonium phosphate, ammonium sulfate, bees wax, bentonite, caffeine, calcium carbonate, calcium chloride, calcium citrate, calcium gluconate, calcium hydroxide, calcium lactate, calcium oxide, calcium phosphate, caramel, carnauba wax, citric acid, dextrans, ethyl formate, glutamic acid, glutamic acid hydrochloride, glyceryl monostearate, lecithin, magnesium carbonate, magnesium hydroxide, magnesium oxide, magnesium stearate, malic acid, methylcellulose, monoammonium glutamate, monopotassium glutamate, papain, potassium acid tartrate, potassium bicarbonate, potassium carbonate, potassium citrate, potassium hydroxide, potassium sulfate, rennet, silica aerogel, sodium acetate, sodium acid pyrophosphate, sodium aluminum phosphate, sodium bicarbonate, sodium carbonate, sodium citrate, sodium carboxy-methylcellulose, sodium caseinate, sodium citrate, sodium hydroxide, sodium pectinate, sodium phosphate, sodium potassium tartrate, sodium sesquicarbonate, sodium tripolyphosphate, succinic acid, tartaric acid, triacetin and triethyl citrate.

Other suitable nucleation particles include nanoparticles composed of a benign material such as sodium ascorbate, calcium carbonate, sodium acetate, glycine, mannose, or calcium citrate.

As used herein, “nanoparticle” refers to a microscopic particle whose size in nanometers (nm) includes a maximum linear dimension of 500 nanometers. As used herein, linear dimension refers to the distance between any two points on a nanoparticle or nucleation particle as measured in a straight line.

Most therapeutic agent delivery stent coatings have thicknesses in the range of 2-20 microns. In order to prevent nucleation particles from roughening or protruding from a coated surface, the nucleation particles added to a coating formulation have a maximum linear dimension of 2 microns. In one aspect of the invention the nucleation particles are no larger than 1/10 the final coating thickness, e.g., 0.2-2.0 microns. Smaller nucleation particles are also encompassed by the present invention including nucleation particles having a maximum linear dimension of 10 nanometers.

In addition to the size of nucleation particles added to the coating formulation, the amount of nucleation particles added will affect the final coating characteristics. In various aspects, the amount of nucleation particles added to a coating formulation is on the order of 0.001% to 25% by weight of the therapeutic agent to be crystallized. In agent eluting coatings, the fraction of the coating which is agent can range from 10% to 90% (w/w).

The ability of nucleation particles to act as a seed for crystallization of an agent is a function of the surface area of the added nucleation particles. For a given mass of nucleation particles, the surface area increases by 1/D (D=diameter). Thus, a population of small particles has a much greater surface area, which is why low nucleation particle loadings are effective. In contrast, loadings of nucleation particles greater than 25% by weight of the therapeutic agent to be crystallized are less desirable, as this can impact the amount of agent which can be loaded as well as the mechanical properties of the coating.

The number of nucleation particles added to the coating formulation affects the crystallization process as well. A large number of added nucleation particles will induce rapid crystallization of a therapeutic agent. This rapid crystallization leads to a large number of relatively small crystals, which is preferable to a relatively small number of large crystals. Smaller crystals are preferred for several reasons. First, agent crystals act as a non-reinforcing filler for the polymer since they have little mechanical interaction with the polymer matrix. Consequently, large crystals serve to weaken the coating. Second, large crystals can create large discrete weak zones for fracture planes, which can act as points for coating failure under stress. Third, small crystals promote uniform agent release from the coating surface which is generally desired to avoid local high concentrations of agent which may lie out of the therapeutic range. Fourth, small agent crystals that may form on the surface of a device (<8 microns) pose less of an embolization hazard than larger crystals.

Similarly, the type of nucleation particles added to a coating formulation will affect the characteristics of drug crystallization. For example, the addition of a select nucleation particle to a formulation containing dexamethasone, PVDF-HFP and an appropriate solvent will induce the dexamethasone to crystallize in a rapid, uniform manner.

Because extraneous particles and impurities can also serve as nuclei for crystal formation, coating formulations are filtered. After this filtration step, insoluble nucleation particles of the invention are added, thereby more accurately and effectively controlling the amount of crystallization in the final coating.

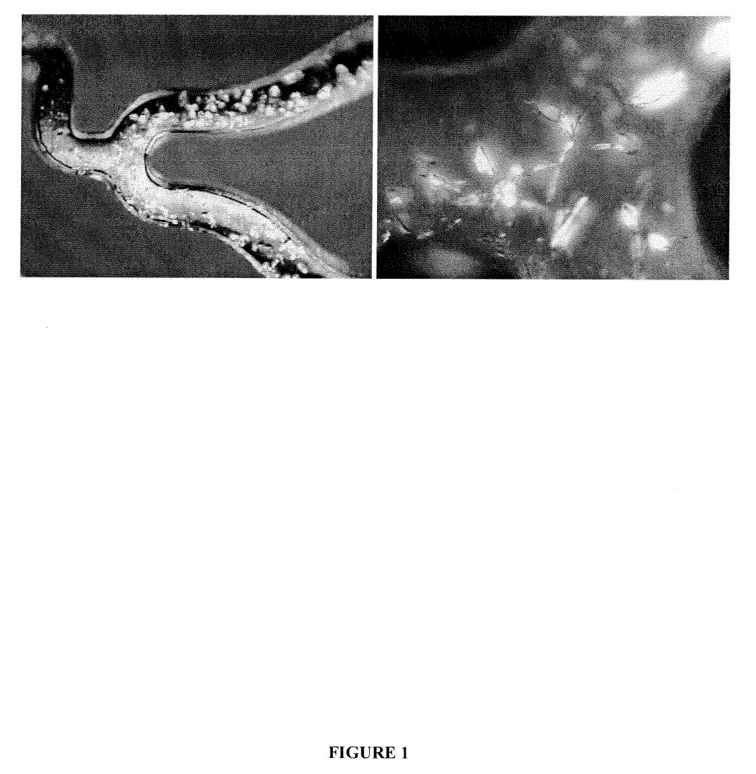

It is to be understood that the method of the present invention can be used to make drug eluting stents with any number of drugs coated on the medical device in crystalline and optionally non-crystalline form. For example, it is possible to produce a stent coated with dexamethasone and an “olimus” drug, e.g., everolimus, in which case the dexamethasone would be uniformly crystallized throughout the coating while everolimus would be present in the coating in a noncrystalline form, as shown in FIG. 1.

In some aspects of the invention, the crystallized therapeutic agent enhances the stability of the coating on an implantable medical device during aging. When an agent is dissolved in a polymer, individual agent molecules can be exposed to potential reactants such as water and oxygen. When an agent is present as crystalline particles, however, agent within the crystal is much better protected against potential reactants, since diffusion of reactants into such a crystal is very slow.

Aqueous-based coating formulations are also encompassed by the present invention. When the solvent in the coating formulation is polar the polymer and therapeutic agents are chosen accordingly. Whether the coating formulation is aqueous-based or organic solvent-based, nucleation particles that are insoluble in the coating formulations are used.

Another aspect of the present invention relates to a method for treating or preventing a vascular disease. The method involves providing an implantable medical device of the invention and implanting the implantable medical device in a vessel of a patient in need thereof.

Methods of implanting a medical device in a vessel are known to those skilled in the art.

The vascular disease to be treated can be atherosclerosis, restenosis, vulnerable plaque or peripheral arterial disease.

As used herein, a “patient” refers to any organism that can benefit from the administration of a therapeutic agent. In particular, patient refers to a mammal such as a cat, dog, horse, cow, pig, sheep, rabbit, goat or a human being.

As used herein, “treating” refers to the administration of a therapeutically effective amount of a therapeutic agent to a patient known or suspected to be suffering from a vascular disease.

As used herein, “known” to be afflicted with a vascular disease refers first to a condition that is relatively readily observable and or diagnosable. An example, without limitation, of such a disease is atherosclerosis, which is a discrete narrowing of a patient's arteries. Restenosis, on the other hand, while in its latter stages, like atherosclerosis, is relatively readily diagnosable or directly observable, may not be so in its nascent stage. Thus, a patient may be “suspected” of being afflicted or of being susceptible to affliction with restenosis at some time subsequent to a surgical procedure to treat an atherosclerotic lesion.

An atherosclerotic lesion refers to a deposit of fatty substances, cholesterol, cellular waste products, calcium and/or fibrin on the inner lining or intima of an artery.

Restenosis refers to the re-narrowing or blockage of an artery at or near the site where angioplasty or another surgical or interventional procedure was previously performed to remove a stenosis.

Vulnerable plaque on the other hand is quite different from either atherosclerosis or restenosis and would generally come under the designation of a “suspected” affliction. This is because vulnerable plaque occurs primarily within the wall of a vessel and does not cause prominent protrusions into the lumen of the vessel. It is often not until it is “too late,” i.e., until after a vulnerable plaque has broken and released its components into the vessel, that its presence is even known. Numerous methods have and are being investigated for the early diagnosis of vulnerable plaque but to date none have proven suitable for widespread application.

As used herein, “peripheral arterial disease” refers to a condition similar to coronary artery disease and carotid artery disease in which fatty deposits build up in the inner linings of the artery walls thereby restricting blood circulation, mainly in arteries leading to the kidneys, stomach, arms, legs and feet.

As used herein, “therapeutically effective amount” refers to the amount of therapeutic agent that has a beneficial effect, which may be curative or palliative, on the health and well-being of a patient with regard to a vascular disease with which the patient is known or suspected to be afflicted.

The amount of therapeutic agent will depend on the required minimum effective concentration (MEC) of the agent and the length of time over which it is desired that the MEC be maintained. For most therapeutic agents the MEC will be known to, or readily derivable by, those skilled in the art from the literature. For experimental therapeutic agents or those for which the MEC by localized delivery is not known, such can be empirically determined using techniques well-known to those skilled in the art.

As used herein, “disease locale” refers to any location within a patient's body where abnormal physiological conditions exist.

As used herein, “vascular disease locale” refers to the location within a patient's body where an atherosclerotic lesion(s) is present, where restenosis may develop, the site of vulnerable plaque(s) or the site of a peripheral arterial disease.

After implantation of the medical device in a patient, the crystallized therapeutic agent is uniformly released over time, thereby providing a means for the localized treatment of a vascular disease.

Similarly, the non-soluble nucleation particles, which are chosen to be non-toxic, can be released over time, especially if they are incorporated into a biodegradable polymer. Nucleation particles composed of water soluble substances will dissolve almost instantly in the aqueous environment of the body upon release. In cases where the particles dissolve more slowly, the particles are chosen so that their presence in the vasculature will not cause any adverse health affects. When the particles are not directly released, exposure of the coating to the in vivo environment will cause the particles to slowly dissolve in the coating then diffuse through the polymer matrix to be released into the vasculature.

Another aspect of the present invention relates to a method for controlling the release rate of a therapeutic agent from an implantable medical device. The method involves providing an implantable medical device, coating the implantable medical device with a formulation comprising a solvent, one or more polymers dissolved in the solvent, one or more crystallizable therapeutic agents dissolved in the solvent and a plurality of non-soluble nucleation particles suspended in the solvent and drying the coating.

Suitable implantable medical devices are described above. Suitable solvents are described above. Suitable crystallizable therapeutic agents are described above and preferably include dexamethasone, clobestasol, momentasone, dexamethasone acetate, cortisone, prednisone, prednisolone or betamethasone.

As described above, the non-soluble nucleation particles can include a pharmaceutical excipient, a biodegradable polymer or a GRAS material and have a maximum linear dimension of 2 microns.

In this aspect of the invention, the crystallized therapeutic agent is uniformly released from the coated implantable medical device after implantation.

### EXAMPLES

### Example 1

### Formation Of A Stent Coating Containing A Crystallized Therapeutic Agent

Primer layer: Poly(n-butyl methacrylate) (PBMA) was dissolved in 70:30 acetone:cyclohexanone (w:w) to give a 2% by weight polymer solution. An external air-assisted atomizing spray nozzle, i.e., an EFD 780S spray nozzle with a VALVEMATE 7040 control system, manufactured by EFD, Inc., East Providence, R.I., was used to spray the polymer solution onto the stent. During the process of applying the composition, the stent was rotated about its longitudinal axis, at a speed of 150 rpm. The stent was also moved linearly along the same axis at a speed of 6 mm/sec during the application.

The 2% solution of the polymer was applied to a 12-mm VISION™ stent (available from Abbott Vascular Corporation) in a series of 5-second passes, to deposit 6 μg of coating per spray pass. Between the spray passes, the stent was dried for 10 seconds using a flow of air at ambient temperature. Six spray passes were applied, followed by baking of the primer layer at 80° C. for 30 minutes, thereby forming a 51 μg PBMA primer layer.

Drug Containing Layer: A mixture was prepared that consisted of, by weight, 2% of poly(vinylidene fluoride-co-hexafluoropropylene), 0.23% of zotarolimus, 0.115% of dexamethasone, and 97.66% 30:70 acetone:cyclohexanone (w:w). The same apparatus used to spray the primer layer on the stent was used to apply the drug layer. Seventy spray passes were performed, at 10 μg/pass, to form a drug-polymer layer. This was followed by drying the drug-polymer layer at 50° C. for 1 hour to yield a 672 μg drug-polymer reservoir layer.

### Example 2

### Formation Of A Stent Coating Containing A Crystallized Therapeutic Agent

Primer layer: Poly(n-butyl methacrylate) is dissolved in 70:30 acetone:cyclohexanone (w:w) to give a 2% by weight polymer solution. An external air-assisted atomizing spray nozzle, as described above, is used to spray the polymer solution onto the stent. During the process of applying the composition, the stent is rotated about its longitudinal axis, at a speed of 150 rpm. The stent is also moved linearly along the same axis at a speed of 6 mm/sec during the application.

The 2% solution of the polymer is applied to a 12-mm VISION™ stent in a series of 5-second passes, to deposit 6 μg of coating per spray pass, as described above. Between the spray passes, the stent is dried for 10 seconds using a flow of air at ambient temperature. Six spray passes are completed, followed by baking the primer layer at 80° C. for 30 minutes, thereby forming a 51 μg PBMA primer layer.

Drug Containing Layer: A mixture is prepared that consists of, by weight, 2% of poly(vinylidene fluoride-co-hexafluoropropylene), 0.166% of everolimus, 0.333% of dexamethasone, 0.033% of lactose nanoparticles (averaging approximately 0.5 microns particle size), and 97.47% of 30:70 acetone:cyclohexanone (w:w). The same apparatus used to spray the primer layer on the stent is used to apply the drug layer. Eighty spray passes are performed, at 12 μg/pass, to form a drug-polymer layer. This is followed by drying the drug-polymer layer at 50° C. for 1 hour to yield a 960 μg drug-polymer reservoir layer.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A method of crystallizing a therapeutic agent in a coating on an implantable medical device, comprising:
providing an implantable medical device;
providing a coating formulation comprising a solvent, one or more polymers dissolved in the solvent, one or more crystallizable therapeutic agents dissolved in the solvent and a plurality of non-soluble nucleation particles suspended in the solvent;
coating the implantable medical device with the coating formulation; and
drying the coating.

2. The method according to claim 1, wherein the implantable medical device comprises a stent.

3. The method according to claim 1, wherein the solvent is an organic solvent.

4. The method according to claim 1, wherein the one or more crystallizable therapeutic agents are selected from the group consisting of an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist and an antioxidant.

5. The method according to claim 4, wherein the anti-inflammatory agent is dexamethasone, clobestasol, momentasone, dexamethasone acetate, cortisone, prednisone, prednisolone or betamethasone.

6. The method according to claim 1, wherein the plurality of non-soluble nucleation particles comprise a pharmaceutical excipient, a biodegradable polymer or a GRAS material.

7. The method according to claim 6, wherein the plurality of non-soluble nucleation particles dissolve upon release from the coating or dissolve within the coating.

8. The method according to claim 7, wherein the plurality of non-soluble nucleation particles are non-toxic.

9. The method according to claim 1, wherein the plurality of non-soluble nucleation particles have a maximum linear dimension of 2 microns.

10. The method according to claim 9, wherein the plurality of non-soluble nucleation particles have a maximum linear dimension of 300 nanometers.

11. The method according to claim 10, wherein the plurality of non-soluble nucleation particles have a maximum linear dimension of 100 nanometers.

12. The method according to claim 11, wherein the plurality of non-soluble nucleation particles have a maximum linear dimension of 10 nanometers.

13. The method according to claim 1, wherein the plurality of non-soluble nucleation particles have a maximum linear dimension no greater than 1/10 the final thickness of the coating.

14. The method according to claim 1, wherein the weight of nucleation particles added to the coating formulation is less than 25 percent of the weight of crystallizable therapeutic agent added to the coating formulation.

15. The method according to claim 1, wherein the crystallized therapeutic agent enhances the stability of the coated implantable medical device during aging.

16. The method according to claim 1, wherein the crystallized therapeutic agent is uniformly released from the coated implantable medical device after implantation.

17. A method of treating or preventing a vascular disease comprising:
providing an implantable medical device made according to the method of claim 1; and
implanting the implantable medical device in a vessel of a patient in need thereof.

18. The method according to claim 17, wherein the vascular disease comprises atherosclerosis, restenosis, vulnerable plaque or peripheral arterial disease.

19. A method for controlling the release rate of a therapeutic agent from an implantable medical device comprising:
providing an implantable medical device;
coating the implantable medical device with a formulation comprising a solvent, one or more polymers dissolved in the solvent, one or more crystallizable therapeutic agents dissolved in the solvent and a plurality of non-soluble nucleation particles suspended in the solvent; and
drying the coating.

20. The method according to claim 19, wherein the implantable medical device comprises a stent.

21. The method according to claim 19, wherein the solvent is an organic solvent.

22. The method according to claim 19, wherein the one or more crystallizable therapeutic agents are selected from the group consisting of an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist and an antioxidant.

23. The method according to claim 19, wherein the plurality of non-soluble nucleation particles comprise a pharmaceutical excipient, a biodegradable polymer or a GRAS material.

24. The method according to claim 19, wherein the plurality of non-soluble nucleation particles have a maximum linear dimension of 2 microns.

25. The method according to claim 19, wherein the crystallized therapeutic agent is uniformly released from the coated implantable medical device after implantation.
